(19) United States
(12) Patent Application Publication
(10) Pub. No.: US 2012/0232426 A1
(43) Pub. Date: Sep. 13, 2012

(54) APPARATUS AND METHODS FOR ADVANCING A WIRE GUIDE

(75) Inventor: Spencer Leiter, Bloomington, IN (US)

(73) Assignee: Cook Medical Technologies LLC, Bloomington, IN (US)

(21) Appl. No.: 13/509,902

(22) PCT Filed: Oct. 29, 2010
(86) PCT No.: PCT/US10/54619

§ 371 Date: May 15, 2012

Related U.S. Application Data
(60) Provisional application No. 61/261,848, filed on Nov. 17, 2009.

(51) Int. Cl. A61M 25/09

(52) U.S. Cl. 600/585

(57) ABSTRACT

Apparatus and methods for advancing a wire guide (70) in either proximal or distal directions. The apparatus comprises a catheter (30) having proximal and distal (40) regions and a wire guide lumen (32) disposed therebetween. A cavity (60) is disposed in the distal region of the catheter, such that the cavity is in communication with a portion of the wire guide lumen. A wire guide is adapted to be disposed through the wire guide lumen and the cavity. At least one actuation mechanism (50) is disposed in the cavity and adapted to at least partially circumferentially surround the wire guide. The actuation mechanism may selectively engage the wire guide to effect movement of the wire guide in at least one direction with respect to the catheter.

### BACKGROUND

The present invention relates generally to apparatus and methods for treating vascular conditions, and more specifically, to apparatus and methods for advancing a wire guide.

Atherosclerosis and other occlusive diseases are prevalent among a significant portion of the population. In such diseases, atherosclerotic plaque forms within the walls of the vessel and blocks or restricts blood flow through the vessel. Atherosclerosis commonly affects the coronary arteries, the aorta, the iliofemoral arteries and the carotid arteries. Several serious conditions may result from the restricted blood flow, such as ischemic events.

Various procedures are known for treating stenoses in the arterial vasculature, such as the use of balloon angioplasty and stenting. During a balloon angioplasty procedure, a catheter having a deflated balloon attached thereto is positioned across a constricting lesion, and the balloon then is inflated to widen the lumen to partially or fully restore patency to the vessel.

Stenting involves the insertion of a usually tubular member into a vessel, and may be used alone or in conjunction with an angioplasty procedure. Stents may be self-expanding or balloon expandable. Self-expanding stents typically are delivered into a vessel within a delivery sheath, which constrains the stent prior to deployment. When the delivery sheath is retracted, the stent is allowed to radially expand to its predetermined shape. If the stent is balloon expandable, the stent typically is loaded onto a balloon of a catheter, inserted into a vessel, and the balloon is inflated to radially expand the stent.

Generally, during each of the foregoing interventional procedures, a wire guide is inserted into a patient's vessel, e.g., under fluoroscopic guidance. The wire guide then is advanced toward a target site in a patient's vasculature. For example, the distal end of the wire guide may be advanced through a stenosis. Then, various medical components, such as a balloon catheter and/or stent, may be distally advanced over the wire guide to the target site.

Various wire guides comprise flexible distal regions to facilitate navigation through the tortuous anatomy of a patient's vessel, but such flexible distal regions may be difficult to be advanced through an occlusion. However, if the distal region of the wire guide is too rigid, then it may not be flexible enough to navigate the tortuous anatomy.

Further, it may be difficult to advance both relatively flexible and relatively rigid wire guides through an occlusion, particularly a narrow or hardened stenosis, by manually pushing from the proximal end of the wire guide. It also may be difficult to achieve incremental positioning of the wire guide on a small scale, such as millimeters and nanometers, by manual advancement of the wire guide.

Therefore, it would be desirable to provide apparatus and methods for achieving enhanced incremental positioning of the wire guide and facilitating advancement of the wire guide, for example, through narrowed or hardened stenoses.

### SUMMARY

The present embodiments provide apparatus and methods for advancing a wire guide in either proximal or distal directions. In one embodiment, the apparatus comprises a catheter having proximal and distal regions and a wire guide lumen disposed therebetween. A cavity is disposed in the distal region of the catheter and placed in communication with a portion of the wire guide lumen. For example, the wire guide lumen may have proximal and distal portions in communication with the cavity, such that a wire guide may be disposed through the proximal portion of the wire guide lumen, through the cavity, and then through the distal portion of the wire guide lumen.

At least one actuation mechanism is disposed in the cavity and adapted to at least partially circumferentially surround the wire guide. The actuation mechanism may be configured to selectively engage the wire guide to effect movement of the wire guide in at least one direction with respect to the catheter, and preferably advance the wire guide in both proximal and distal directions. Further, the actuation mechanism may comprise a relaxed state in which the wire guide is not engaged to allow manual advancement of the wire guide through the wire guide lumen and the cavity.

In one embodiment, the actuation mechanism comprises first, second and third actuators disposed within the cavity and configured to be independently actuated to effect movement of the wire guide. The first actuator may be disposed adjacent to the second actuator, and the second actuator may be disposed adjacent to the third actuator. The first and third actuators may comprise toroidal actuators that are adapted to selectively clamp and release the wire guide, and the second actuator may comprise a linear actuator adapted to selectively move the first and third actuators in proximal and distal directions. Accordingly, by independently actuating the first, second and third actuators in a desired sequence, proximal and distal advancement of the wire guide may be achieved.

The first, second and third actuators may comprise piezoelectric components. The catheter may comprise at least one auxiliary lumen that is configured to receive at least one control wire for transmitting an electric signal to at least one of the first, second and third actuators. In one exemplary method, the catheter may be used to facilitate advancement of a wire guide through a vascular condition, such as a stenosis.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side-sectional view of a catheter that may be used to advance a wire guide.

FIG. 2 is a cross-sectional view of the catheter along line A-A of FIG. 1.

FIGS. 3A-3H are schematic diagrams illustrating an exemplary use of an actuation mechanism disposed in the catheter of FIG. 1.

FIGS. 4-9 are side-sectional views illustrating exemplary method steps that may be used to advance a wire guide through an occlusion.

### DETAILED DESCRIPTION OF THE PREFERRED EMBODIMENTS

In the present application, the term “proximal” refers to a direction that is generally toward a physician during a medical procedure, while the term “distal” refers to a direction that is generally toward a target site within a patient's anatomy during a medical procedure.

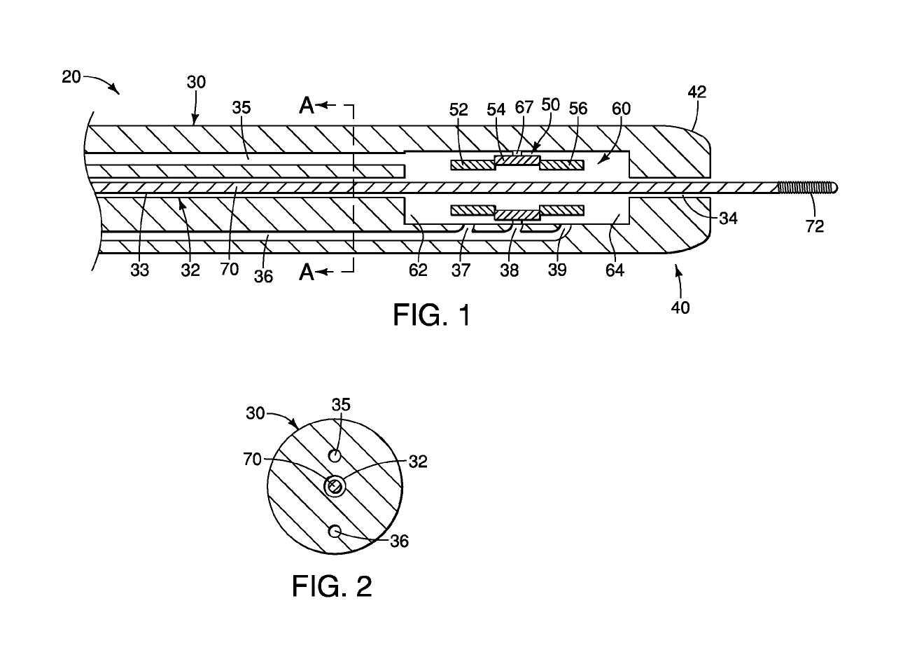

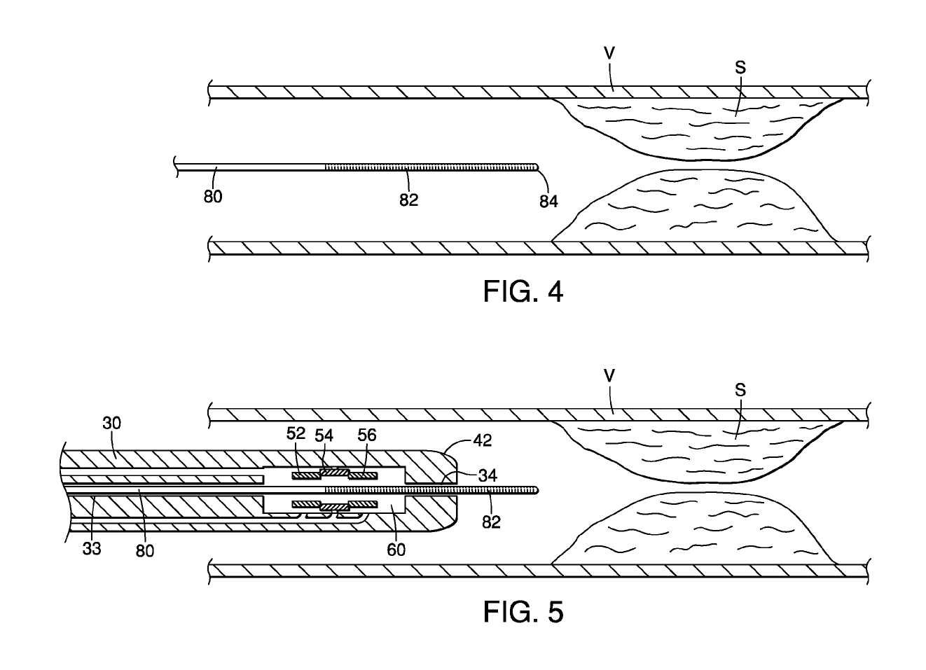

Referring now to FIG. 1, an apparatus 20 suitable for advancing a wire guide 70 is shown and described. The apparatus 20 comprises a catheter 30, which has proximal and distal regions and a wire guide lumen 32 disposed therebetween. The wire guide lumen 32 is adapted to receive one or more different wire guides, such as a relatively stiff wire guide 70 (see FIG. 1 and FIGS. 7-8) and a relatively flexible wire guide 80 (see FIGS. 4-5), and further allows movement of the wire guides therethrough.

The catheter 30 may comprise a flexible, tubular member that may be formed from one or more semi-rigid polymers. For example, the catheter may be manufactured from polyurethane, polyethylene, tetrafluoroethylene, polytetrafluoroethylene, fluorinated ethylene propylene, nylon, PEBAX or the like.

As shown in FIG. 1, the catheter 30 further comprises a cavity 60 formed in the distal region 40 of the catheter. The cavity 60 has proximal and distal regions 62 and 64, respectively, each of which may be placed in communication with the wire guide lumen 32. The wire guide lumen 32 therefore may comprise a proximal portion 33 that is disposed proximal to the cavity 60, and further may comprise a distal portion 34 that is disposed distal to the cavity 60, as shown in FIG. 1. Accordingly, a wire guide 70 may extend through the proximal portion 33 of the wire guide lumen 32, through the cavity 60, through the distal portion 34 of the wire guide lumen 32, and subsequently exit through the distal region 40 of the catheter 30.

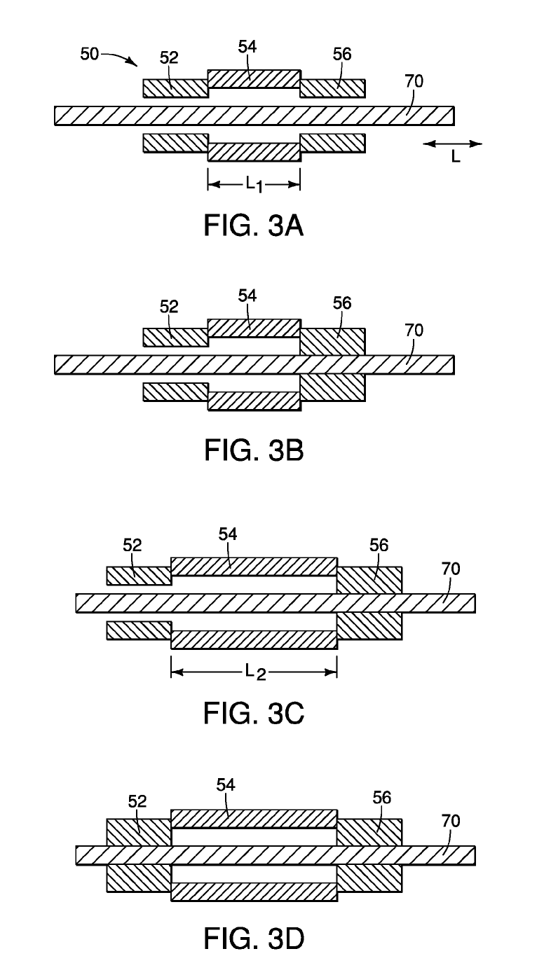

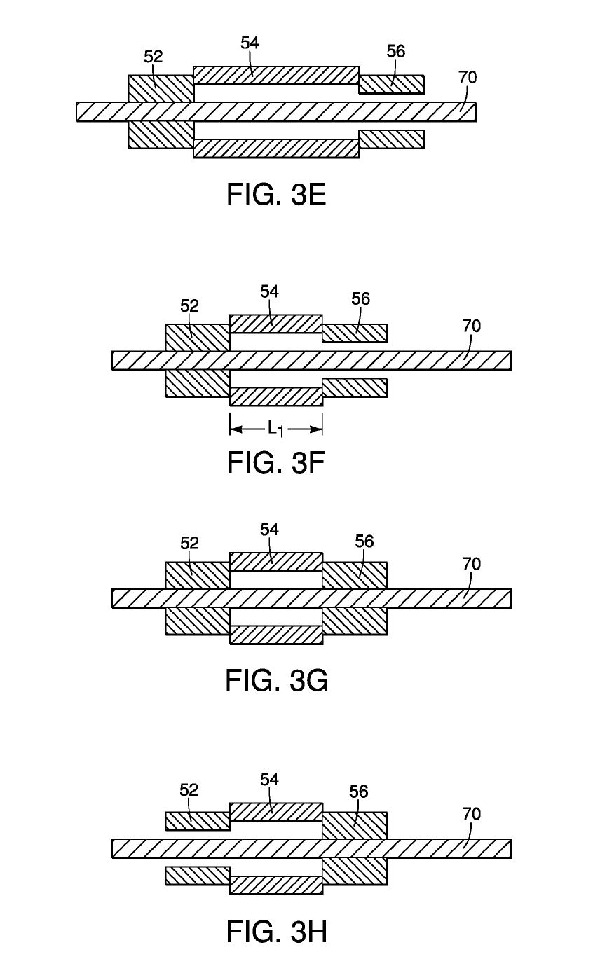

Referring still to FIG. 1, at least one actuation mechanism 50 may be disposed in the cavity 60. As explained further below, the actuation mechanism 50 may at least partially circumferentially surround the wire guide 70, and further may selectively engage the wire guide 70 to effect movement of the wire guide 70 in a proximal or distal direction. In one embodiment, shown in FIG. 1 and described with respect to FIGS. 3A-3H below, the actuation mechanism 50 may comprise a series of first, second and third actuators 52, 54 and 56, each of which is disposed within the cavity 60 and at least partially circumferentially surrounds the wire guide 70.

In one embodiment, the three actuators 52, 54 and 56 may operate in conjunction with one another to facilitate movement of the wire guide 70 in either proximal or distal directions. For example, the first and third actuators 52 and 56 may comprise toroidal actuators that are configured to selectively and independently clamp down upon the wire guide 70 upon actuation. The second actuator 54 may comprise a linear actuator that is configured to selectively and independently expand in a linear direction along a longitudinal axis L, as described further below with respect to FIGS. 3A-3H.

The three actuators 52, 54 and 56 may be physically coupled to one another, but may be actuated independently of one another. In a preferred embodiment, a distal end of the first actuator 52 is coupled to a proximal end of the second actuator 54, while a distal end of the second actuator 54 is coupled to a proximal end of the third actuator 56. As shown in FIG. 1 and explained in FIG. 3A, when all three of the actuators 52, 54 and 56 are in their relaxed, non-actuated states, open spaces may be formed at the proximal and distal regions 62 and 64 of the cavity 60. The open spaces in the cavity 60 may accommodate subsequent longitudinal movement of the actuators, as explained below with respect to the actuation steps described in FIGS. 3A-3H.

The second actuator 54 may be coupled to a motor 67, which may be disposed near a central region of the cavity 60, as shown in FIG. 1. The motor 67 may comprise an Inchworm® motor manufactured by EXFO Burleigh Products Group, Inc. of Victor, N.Y. A variable-rate staircase voltage may be applied to the second actuator 54, causing it to change length in discrete steps on the order of nanometers, as explained below.

In a preferred embodiment, the first, second and third actuators 52, 54 and 56 may comprise piezoelectric actuators, which therefore undergo a dimensional change when an electrical signal is applied. The first and third actuators 52 and 56 may be provided in accordance with clamping piezoelectric actuators manufactured by EXFO Burleigh Products

Group, Inc. of Victor, N.Y, while the second actuator 54 may be provided in accordance with a linear actuator manufactured by the same company. The dimensional change of the first, second and third actuators 52, 54 and 56 may be proportional to the voltage or current applied to the actuators. An electronic controller may be provided and configured to be operated by a physician or assistant to output the desired voltage or current waveforms.

Electronic signals may be provided to the first, second and third actuators 52, 54 and 56 using one or more control wires (not shown), which may be disposed through an auxiliary lumen 35 and/or an auxiliary lumen 36. As shown in FIG. 1, the auxiliary lumen 35 may be placed in direct communication with the proximal region 62 of the cavity 60, while the auxiliary lumen 36 may transition into one or more outlets 37, 38 and 39, which may be placed in close proximity to the first, second and third actuators 52, 54 and 56, respectively.

The motor 67 may be coupled to the catheter 30 such that the motor 67 fixes the longitudinal positioning of the first, second and third actuators 52, 54 and 56 within the cavity 60. The motor 67 may be secured to the second actuator 54, and further secured to an interior wall of the catheter 30 to maintain the positioning of the actuation mechanism 50 within the cavity 60.

Referring now to FIGS. 3A-3H, a schematic use of the actuation mechanism 50 of

FIG. 1 is described. As noted above, the first, second and third actuators 52, 54 and 56 may at least partially circumferentially surround the wire guide 70. Further, the distal end of the first actuator 52 may be coupled to the proximal end of the second actuator 54, while the distal end of the second actuator 54 may be coupled to the proximal end of the third actuator 56. In this manner, each of the first, second and third actuators 52, 54 and 56 may be coupled together and move in the manner described below.

The first, second and third actuator 52, 54 and 56 each have first states, in which they are relaxed, and second states, in which they are actuated. Each of the actuators 52, 54 and 56 may be operated independently to be transformed from the first state to the second state by selectively applying and removing an electric signal to each of the actuators, as explained below.

In FIG. 3A, the first, second and third actuators 52, 54 and 56 are shown in their relaxed states. The first, second and third actuators 52, 54 and 56 may at least partially circumferentially surround, but do not engage, the wire guide 70 when in their relaxed states. Accordingly, the wire guide 70 may pass longitudinally through the series of actuator 52, 54 and 56. Further, in its relaxed state, the second actuator 54 has a reduced longitudinal length L₁, as shown in FIG. 3A.

Referring now to FIG. 3B, in a first step for advancing the wire guide 70 distally with respect to the catheter 30, the third actuator 56 may be actuated to engage an outer surface of the wire guide 70. The third actuator 56 may be actuated by applying an electric signal via a control wire disposed through either the auxiliary lumen 35 or the auxiliary lumen 36. At this time, the third actuator 56 undergoes a dimensional change that causes it to clamp down to securely engage the wire guide 70.

In a next step, shown in FIG. 3C, the second actuator 54 may be actuated to distally advance the wire guide 70. More specifically, the second actuator 54 may be actuated by applying an electric signal via a control wire disposed through either auxiliary lumen 35 or auxiliary lumen 36 and coupled to the motor 67, thereby increasing the length of the second actuator 54 to a longitudinal length L₂, as shown in FIG. 3C. The second actuator 54 may expand longitudinally in both proximal and distal directions with respect to the catheter 30. Since only the third actuator 56 is securely clamped to the wire guide 70, and the third actuator is advanced in a distal direction by the longitudinal expansion of the second actuator 54, the wire guide 70 also may be advanced in a distal direction, as shown in FIG. 3C.

Referring now to FIG. 3D, with the second and third actuators 54 and 56 still in their respective actuated states, the first actuator 52 may be actuated to engage an outer surface of the wire guide 70. The first actuator 52 may be actuated by applying an electric signal via a control wire disposed through either auxiliary lumen 35 or auxiliary lumen 36, which causes the first actuator 52 to become clamped against the wire guide 70 at a location proximal to the second actuator 54.

Subsequently, the electric signal applied to the third actuator 56 may be removed to cause the third actuator 56 to assume a relaxed state, as shown in FIG. 3E. In the relaxed state, the third actuator 56 does not engage the wire guide 70. Then, the electric signal applied to the second actuator 54 may be removed to cause the second actuator 54 to assume a relaxed state in which it returns to a reduced longitudinal length L₁, as shown in FIG. 3F. At this time, since only the first actuator 52 is securely clamped to the wire guide 70, the first actuator 52 will be advanced in a distal direction by the longitudinal contraction of the second actuator 54. Accordingly, the wire guide 70 is advanced further in a distal direction, as shown in FIG. 3F.

Finally, the third actuator 56 may be actuated to engage an outer surface of the wire guide 70, as shown in FIG. 3G, and then the electric signal applied to the first actuator 52 may be removed to cause the first actuator 52 to assume a relaxed state, as shown in FIG. 3H. In the relaxed state, the first actuator 52 does not engage the wire guide 70. At this time, each of the actuators are in the position shown in FIG. 3B, and the series of actuation steps between FIGS. 3B-3H may be repeated to continue to advance the wire guide 70 in a distal direction.

The process described in FIGS. 3B-3H may be repeated as frequently as desired. For example, the process may be repeated thousands of times per second. If piezoelectric actuators are employed, they may be operated over millions of cycles without significant wear or deterioration. If the first, second and third actuators 52, 54 and 56 comprise piezoelectric actuators, then incremental positioning may be obtained on a scale of nanometers. Accordingly, it may be possible to move the wire guide 70 on a nanometer scale over several millimeters of continuous motion. Advantageously, the actuation mechanism 50 therefore may enable higher resolution positioning of the wire guide 70, for example, relative to pushing a wire guide manually.

Moreover, the wire guide 70 may be moved incrementally in a proximal direction by reversing one or more of the steps described for FIG. 3B-3H. For example, in a first step to effect proximal movement of the wire guide 70, the first actuator 52 may be actuated in FIG. 3B, in lieu of the third actuator 56. Then, when the second actuator 54 is actuated to expand in a longitudinal direction, as explained in FIG. 3C, the first actuator 52 will move the wire guide 70 in a proximal direction. In this manner, proximal movement of the wire guide 70 may be achieved, in lieu of the distal movement explained above.

As noted above, the first, second and third actuators 52, 54 and 56 preferably comprise piezoelectric actuators, which therefore undergo a dimensional change when an electrical signal is applied. The dimensional change may be proportional to the voltage or current applied to the actuators. Accordingly, the provision of a variable voltage to the second actuator 54 may impact the linear change associated with the second actuator 54 and may affect the incremental linear movement of the wire guide 70.

Referring now to FIGS. 4-9, exemplary method steps for using apparatus 20 to advance a wire guide through a vascular condition, such as stenosis S in a vessel V, are shown and described. In a first step, a relatively flexible wire guide 80 may be inserted into the patient's vessel V and positioned proximal to the stenosis S. The relatively flexible wire guide 80 may comprise a distal region comprising a coiled member 82 and an atraumatic tip 84, as shown in FIG. 4. The relatively flexible wire guide 80 may be configured to navigate a patient's tortuous vasculature, for example, using fluoroscopic guidance. However, due to its substantially flexible distal region, the relatively flexible wire guide 80 may not be well-suited for traversing stenosis S, particularly if the occlusion is substantially narrow or hardened.

Referring now to FIG. 5, in a next step, the catheter 30 is advanced distally over the relatively flexible wire guide 80 with the actuation mechanism 50 in the relaxed state. More specifically, the first, second and third actuators 52, 54 and 56 do not circumferentially engage the wire guide 80, thereby allowing the catheter 30 to be advanced over the flexible wire guide 80 via the wire guide lumen portions 33 and 34. One or more radiopaque markers (not shown) may be disposed on the catheter 30 to facilitate positioning of the catheter 30 with respect to the stenosis S. Further, the distal region 40 of the catheter 30 may comprise a tapered end 42, which may facilitate insertion of the catheter 30 over the flexible wire guide 80 and into smaller vessels.

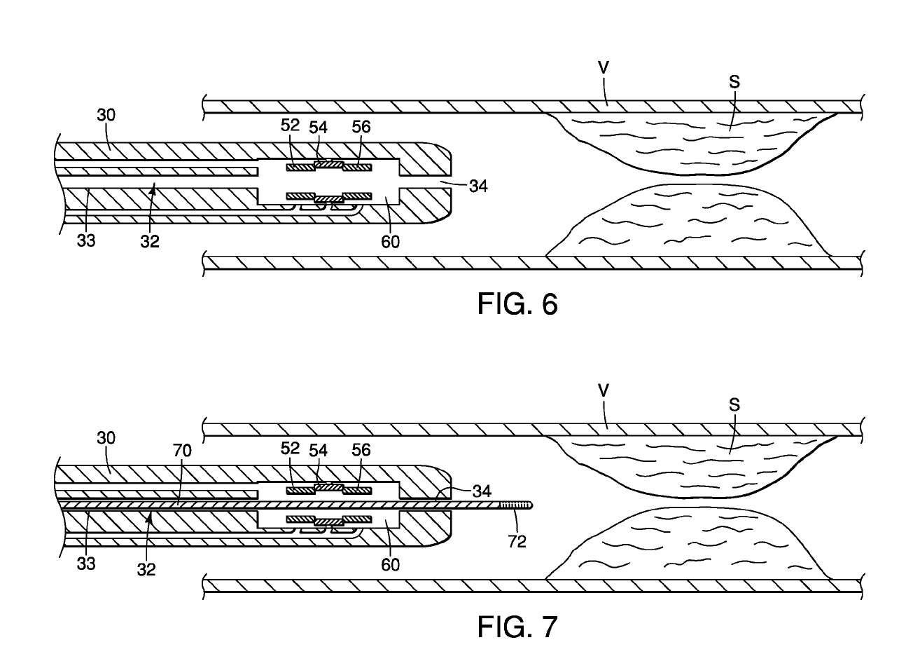

Referring now to FIGS. 6-7, the relatively flexible wire guide 80 may be proximally retracted and withdrawn from the patient's vasculature. At this time, the catheter 30 may be held stationary within the patient's vessel V at a location proximal to the stenosis S, as shown in FIG. 6. In a next step, the relatively stiff wire guide 70 may be loaded through the proximal end of the catheter 30. More specifically, while the catheter 30 is held stationary, the relatively stiff wire guide 70 is advanced distally through the proximal portion 33 of the wire guide lumen 32, through the relaxed actuation mechanism 50 within the cavity 60, then advanced through the distal portion 34 of the wire guide lumen 32. At this time, the relatively stiff wire guide 70 may be manually positioned at a location just proximal to the stenosis S, as shown in FIG. 7.

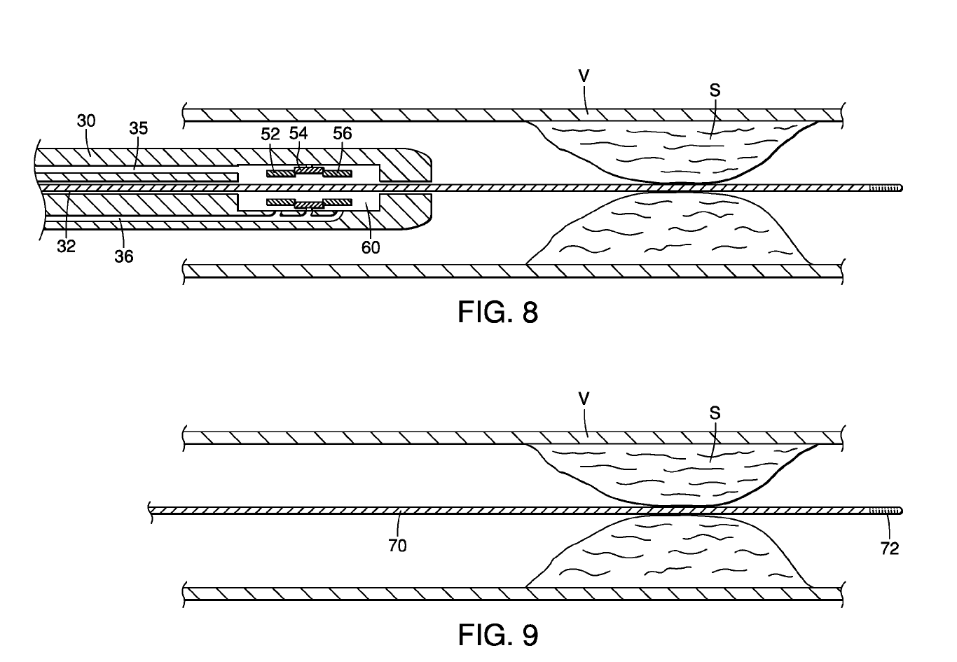

Referring now to FIG. 8, in a next step, the actuation mechanism 50 may be actuated to distally advance the relatively stiff wire guide 70 through the stenosis S, preferably using the method steps described above with respect to FIGS. 3A-3H. If needed, the relatively stiff wire guide 70 may be moved in alternating proximal and distal directions by varying the actuation of the first, second and third actuators 52, 54 and 56, as generally explained above.

Once the relatively stiff wire guide 70 traverses the stenosis S, such that the distal end 72 of the wire guide 70 is distal to the stenosis S, the actuation of each of the first, second and third actuators 52, 54 and 56 may be ceased to release the wire guide 70, as shown in FIG. 8. Subsequently, the catheter 30 may be withdrawn proximally, while the relatively stiff wire guide 70 is held in place, thereby leaving the relatively stiff wire guide 70 within the vessel V, as shown in FIG. 9. With the relatively stiff wire guide 70 disposed through the stenosis S, a second catheter (not shown) may be inserted over the wire guide 70 and into the vessel V to treat the stenosis S. The second catheter may treat the stenosis S using any known technique, including but not limited to performing balloon angioplasty, stenting, delivering a therapeutic agent, and the like. Once the desired therapies have been performed to treat the stenosis S, the second catheter and the relatively stiff wire guide 70 may be removed from the patient's vessel.

Advantageously, the apparatus and methods described above allow a physician to advance a wire guide in proximal and distal directions with increased incremental positioning on the scale of nanometers. Further, since the actuation mechanism 50 is disposed in a cavity 60 positioned at the distal region 40 of the catheter 30, the mechanism for advancing the wire guide may be placed in substantially close proximity to a vascular occlusion. By engaging and advancing a distal region of a wire guide, as opposed to pushing the proximal region of the wire guide outside of the body, an improved positioning and localized force transmission through the stenosis S may be achieved.

It will be apparent that while the embodiments have been described primarily with respect to advancing a wire guide through a stenosis within a vessel, the present embodiments may be used in other applications. Further, the catheter 30 and the wire guides 70 and 80 may employ an over-the-wire arrangement, or alternatively, the catheter 30 may comprise a rapid exchange port disposed in a lateral surface of the catheter 30. Finally, while the present embodiments generally depict an actuation mechanism 50 having first, second and third actuators 52, 54 and 56 that are coupled together and engage the wire guide in a manner shown in FIGS. 3A-3H, greater or fewer actuators may be employed and may operate in different sequences other than those described.

While various embodiments of the invention have been described, it will be apparent to those of ordinary skill in the art that many more embodiments and implementations are possible within the scope of the invention. Accordingly, the invention is not to be restricted except in light of the attached claims and their equivalents. Moreover, the advantages described herein are not necessarily the only advantages of the invention and it is not necessarily expected that every embodiment of the invention will achieve all of the advantages described.

1. An apparatus suitable for advancing a wire guide, the apparatus comprising:
a catheter having proximal and distal regions;
a wire guide lumen disposed between the proximal and distal regions of the catheter, wherein the wire guide lumen is adapted to receive the wire guide;
a cavity disposed in the distal region of the catheter, wherein the cavity is in communication with a portion of the wire guide lumen; and
at least one actuation mechanism disposed in the cavity, wherein the actuation mechanism is adapted to at least partially circumferentially surround the wire guide and selectively engage the wire guide to effect movement of the wire guide in at least one direction with respect to the catheter.

2. The apparatus of claim 1 wherein the wire guide lumen comprises proximal and distal portions, and wherein the wire guide is adapted to be disposed through the proximal portion of the wire guide lumen, through the actuation mechanism in the cavity, and further through the distal portion of the wire guide lumen.

3. The apparatus of claim 1 wherein the actuation mechanism is configured to effect movement of the wire guide in both proximal and distal directions with respect to the catheter.

4. The apparatus of claim 1 wherein the actuation mechanism comprises a relaxed state in which the wire guide is not engaged to allow manual advancement of the wire guide through the cavity.

5. The apparatus of claim 1 wherein the actuation mechanism comprises first, second and third actuators disposed within the cavity and configured to be independently actuated to effect movement of the wire guide.

6. The apparatus of claim 5 wherein the first actuator is disposed adjacent to the second actuator and the second actuator is disposed adjacent to the third actuator,
wherein the first and third actuators comprise toroidal actuators that are adapted to selectively clamp and release the wire guide, and
wherein the second actuator comprises a linear actuator adapted to selectively move the first and third actuators in proximal and distal directions.

7. The apparatus of claim 6 wherein the first, second and third actuators comprise piezoelectric components.

8. The apparatus of claim 5 wherein the catheter comprises at least one auxiliary lumen that is configured to receive at least one control wire for transmitting an electric signal to at least one of the first, second and third actuators.

9. A method suitable for advancing a wire guide, the method comprising:
providing a catheter having proximal and distal regions, a wire guide lumen disposed between the proximal and distal regions, and a cavity disposed in the distal region of the catheter, wherein the cavity is in communication with a portion of the wire guide lumen;
positioning the wire guide with respect to the catheter such that at least a portion of the wire guide is disposed through the cavity and at least a portion of the wire guide is disposed in the wire guide lumen; and
actuating at least one actuation mechanism disposed in the cavity to selectively engage the wire guide and effect movement of the wire guide in at least one direction with respect to the catheter.

10. The method of claim 9 wherein the wire guide lumen comprises proximal and distal portions, the method further comprising disposing the wire guide through the proximal portion of the wire guide lumen, through the actuation mechanism in the cavity, and further through the distal portion of the wire guide lumen.

11. The method of claim 9 further comprising using the actuation mechanism to selectively move the wire guide in both proximal and distal directions.

12. The method of claim 9 further comprising placing the actuation mechanism in a relaxed state in which the wire guide is not engaged to allow manual advancement of the wire guide through the cavity.

13. The method of claim 9 wherein the actuation mechanism comprises first, second and third actuators disposed within the cavity and configured to be independently actuated to effect movement of the wire guide, wherein the first actuator is disposed adjacent to the second actuator and the second actuator is disposed adjacent to the third actuator, and wherein the first, second and third actuators comprise piezoelectric components, the method further comprising:
selectively actuating the first and third actuators to selectively clamp and release the wire guide; and
selectively actuating the second actuator to move the first and third actuators in proximal and distal directions.

14. The method of claim 9 wherein, prior to the step of actuating the at least one actuation mechanism, the method further comprises:
inserting a relatively flexible wire guide into a patient's vessel;
distally advancing the catheter over the relatively flexible wire guide;
proximally retracting and removing the relatively flexible wire guide from the wire guide lumen of the catheter;
distally advancing a relatively stiff wire guide through the wire guide lumen.

15. An apparatus suitable for advancing a wire guide, the apparatus comprising:
a catheter having proximal and distal regions;
a wire guide lumen disposed between the proximal and distal regions of the catheter, wherein the wire guide lumen is adapted to receive the wire guide;
a cavity disposed in the distal region of the catheter, wherein the cavity is in communication with a portion of the wire guide lumen; and
at least one actuation mechanism disposed in the cavity, wherein the actuation mechanism comprises at least first, second and third actuators,
wherein the first, second and third actuators are configured to be independently actuated to selectively move the wire guide in a proximal or distal direction with respect to the catheter.

16. The apparatus of claim 15 wherein the first actuator is disposed adjacent to the second actuator and the second actuator is disposed adjacent to the third actuator,
wherein the first and third actuators comprise toroidal actuators that are adapted to selectively clamp and release the wire guide, and
wherein the second actuator comprises a linear actuator adapted to selectively move the first and third actuators in proximal and distal directions.

17. The apparatus of claim 16 wherein the first, second and third actuators comprise piezoelectric components.

18. The apparatus of claim 15 wherein the actuation mechanism comprises a relaxed state in which the wire guide is not engaged to allow manual advancement of the wire guide through the cavity.

19. The apparatus of claim 15 wherein the wire guide lumen comprises proximal and distal portions, and wherein the wire guide is adapted to be disposed through the proximal portion of the wire guide lumen, through the actuation mechanism in the cavity, and further through the distal portion of the wire guide lumen.

20. The apparatus of claim 15 wherein the catheter comprises at least one auxiliary lumen that is configured to receive at least one control wire for transmitting an electric signal to at least one of the first, second and third actuators.
